(19) United States
(12) Patent Application Publication
(10) Pub. No.: US 2007/0254930 A1
(43) Pub. Date: Nov. 1, 2007

(54) Oral Preparation Having Improved Bioavailability

(75) Inventors: Jei Man Ryu, Kyunggi-do (KR); Soon Ki Cho, Kyunggi-do (KR); Se Hyun Jung, Kyunggi-do (KR); Seung-Kyoo Seong, Kyunggi-do (KR); Eun Hee Cho, Kyunggi-do (KR); Seok Hoon Ahn, Seoul (KR); Yun-Jung Kim, Kyunggi-do (KR)

Correspondence Address: LUCAS & MERCANTI, LLP, 475 PARK AVENUE SOUTH, 15TH FLOOR, NEW YORK, NY, 10016, US

(73) Assignee: DONG WHA PHARM. IND. CO., LTD., Seoul (KR)

(21) Appl. No.: 11/577,469

(22) PCT Filed: Nov. 22, 2005
(86) PCT No.: PCT/KR05/03950

§ 371 Date: Apr. 18, 2007

(30) Foreign Application Priority Data
Nov. 23, 2004 (KR) 10-2004-0096390

(51) Int. Cl. A61K 31/426; A61P 19/10; C07D 277/28

(52) U.S. Cl. 514/365; 548/205

(57) ABSTRACT

The present invention relates to an oral preparation of N-hydroxy-4-{5-[4-(5-isopropyl-2-methyl-1,3-thiazol-4-yl) phenoxy]pentoxyl-benzamidine having improved bioavailability. More particularly, the present invention relates to an oral preparation comprising: N-hydroxy-4-{5-[4-(5-isopropyl-2-methyl-1,3-thiazol-4-yl) phenoxy]pentoxy}-benzamidine or pharmaceutically acceptable salt thereof; and one or more carbonates selected from the group consisting of alkalimetal carbonate, alkalimetal bicarbonate and alkaline earth metal carbonate, and/or one or more disintegrants selected from the group consisting of sodium starch glycolate, carmellose calcium and croscarmellose sodium. The oral preparation according to the present invention inhibits gelation of N-hydroxy-4-{5-[4-(5-isopropyl-2-methyl-1,3-thiazol-4-yl) phenoxy]pentoxy}-benzamidine or pharmaceutically acceptable salt thereof in the early stage of release, which increases dissolution rate and remarkably raises bioavailability.

### TECHNICAL FIELD

The present invention relates to an oral preparation of N-hydroxy-4-5-[4-(5-isopropyl-2-methyl-1,3-thiazol-4-yl)phe noxy]pentoxy}-benzamidine having improved bioavailability.

More particularly, the present invention relates to an oral preparation comprising: N-hydroxy-4-{5-[4-(5-isopropyl-2-methyl-1,3-thiazol-4-yl)phenoxy]pentoxy}-benzamidine of Chemical Formula 1 or pharmaceutically acceptable slat thereof; and one or more carbonates selected from the group consisting of alkali metal carbonate, alkali metal bicarbonate and alkaline earth metal carbonate and/or one or more disintegrating agents selected from the group consisting of sodium starch glycolate, carmellose calcium and croscarmellose sodium.

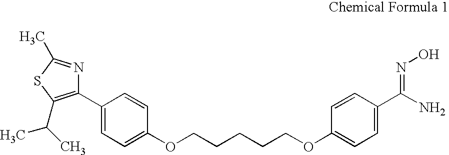

### BACKGROUND ART

The present inventors have disclosed that N-hydroxy-4-{5-[4-(5-isopropyl-2-methyl-1,3-thiazol-4-yl)phenoxy]pentoxy}-benzamidine of Chemical Formula 1 and salts thereof suppress excessive bone absorption by inhibiting the function of osteoclast, thereby having excellent preventive and therapeutic effects on osteoporosis in Korean Patent Laid-Open Publication No. 10-2003-0008654.

The compound of Chemical Formula 1 is poorly water-soluble, highly lipophilic and a weak base which is mostly ionized in a low pH condition such as gastric juice. Accordingly, the water solubility of the compound of Chemical Formula 1 decreases dramatically when a pH of a solution changes from a strongly acidic condition to a weakly acidic or weakly basic condition (pH 3˜pH 7.5). Especially, the compound of Chemical Formula 1 is practically insoluble in aqueous condition, of which pH is about pH 5 or more, and gelates by itself when contacting with water.

With regard to pharmaceutical aspects, in order that the compound of Chemical Formula 1 may show proper drug efficacy as an active ingredient, the pharmaceutical preparation thereof should be quickly disintegrated in the stomach, and the active ingredient should be readily released to be absorbed into the body. In addition, the solubility of the compound of Chemical Formula 1 depends on the pH of aqueous condition. Although the compound is soluble in very strongly acidic aqueous condition such as gastric juice in which the compound is mostly ionized, it is practically insoluble in weakly acidic or neutral aqueous condition. Furthermore, since it gelates by itself when contacting with water, rapid release and effective absorption into the body could not be expected.

Generally, for the purpose of improving the dissolution rate of poorly water-soluble active ingredients, there have been suggested pharmaceutical methods such as reduction of particle size of an active ingredient, polymorphism, amorphous form, spray drying, mixed crushing, solid dispersion into a water-soluble polymer, solvated compound, interaction with additives, etc. Among them, a solid dispersion, in which insoluble active ingredients is dispersed into a pharmaceutically inactive water-soluble polymer, is well known as a method that increases the dissolution rate of an insoluble active ingredient (Albert et al., International Journal of Pharmaceutics, Vol. 104, p169-174, 1994; J. M. Gines et al., International Journal of Pharmaceutics, Vol. 143, p247-253, 1996).

In this regard, the present inventors conducted studies to increase dissolution of the compound of Chemical Formula 1 using the aforementioned methods such as solid dispersion into a water-soluble polymer, spray drying, mixed crushing and amorphous form. However, dissolution was not improved rather suppressed, so satisfactory results were not obtained.

Accordingly, the present inventors performed intensive and thorough study to analyze the causes of slow dissolution rate of the compound and to solve the problems.

The study resulted in the finding that the dissolution rate and bioavailability maybe remarkably increased when the compound of Chemical Formula 1 is formulated with a specific disintegrant such as a starch derivative or cellulose derivatives, carbonate or a mixture of the aforementioned specific disintegrant and carbonate, which led to the present invention.

### DISCLOSURE OF THE INVENTION

Technical Problem

It is an object of the present invention to provide an oral preparation, which has improved bioavailability, comprising N-hydroxy-4-{5-[4-(5-isopropyl-2-methyl-1,3-thiazol-4-yl)phenoxy]pentoxy}-benzamidine or pharmaceutically acceptable salt thereof, and a carbonate and/or a specific disintegrant.

Technical Solution

To achieve the above object, the present invention provides an oral preparation comprising: N-hydroxy-4-{5-[4-(5-isopropyl-2-methyl-1,3-thiazol-4-yl)phenoxy]pentoxy}-benzamidine of Chemical Formula 1 or pharmaceutically acceptable salt thereof; and a carbonate.

In other aspect, the present invention provides an oral preparation comprising: N-hydroxy-4-{5-[4-(5-isopropyl-2-methyl-1,3-thiazol-4-yl)phenoxy]pentoxy}-benzamidine of Chemical Formula 1 or pharmaceutically acceptable salt thereof; and one or more disintegrants selected from the group consisting of sodium starch glycolate, carmellose calcium and croscarmellose sodium.

In a further aspect, the present invention provides an oral preparation comprising: N-hydroxy-4-{5-[4-(5-isopropyl-2-methyl-1,3-thiazol-4-yl)phenoxy]pentoxy}-benzamidine of Chemical Formula 1 or pharmaceutically acceptable salt thereof; a carbonate; and one or more disintegrants selected from the group consisting of sodium starch glycolate, carmellose calcium and croscarmellose sodium. 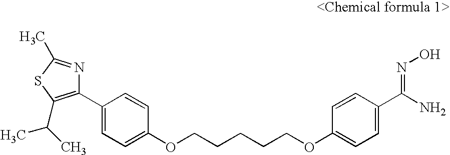
Advantageous Effect

An oral preparation according to the present invention increases the dissolution rate of the compound of Chemical Formula 1 and remarkably enhances the bioavailability of the compound of Chemical Formula 1 by suppressing the gelation of it when contacting with water in the early stage of release. The oral preparation of N-hydroxy-4-{5-[4-(5-isopropyl-2-methyl-1,3-thiazol-4-yl)phenoxy]pentoxy}-benzamidine or pharmaceutically acceptable salt thereof, which have a characteristic of gelating by itself when contacting with water, is formulated with a carbonate and/or with a specific disintegrant in order to prevent the gelation.

In addition to the aforementioned components, an oral preparation according to the present invention may further include one or more pharmaceutically acceptable inorganic excipients such as calcium biphosphate, calcium phosphate or precipitated calcium carbonate. The inorganic excipients not only improve dissolution rate of the compound of Chemical Formula 1, but also act as a calcium supplier for preventing and treating osteoporosis.

### DESCRIPTION OF DRAWINGS

FIG. 1 shows the results of a dissolution test of oral preparations (capsule) according to the present invention;

FIG. 2 shows the results of a dissolution test of oral preparations (tablet) according to the present invention;

FIG. 3 shows the results of a dissolution test of oral preparations according to the present invention containing inorganic or organic excipient; and

FIG. 4 shows the results of bioavailability study of oral preparations according to the present invention.

### BEST MODE

Hereinafter, the present invention will be described in detail.

The present invention relates to an N-hydroxy-4-{5-[4-(5-isopropyl-2-methyl-1,3-thiazol-4-yl)phenoxy]pentoxy}-benzamidine represented by Chemical Formula 1 or, which has the characteristics of pH-dependent solubility, strong electrostatic attraction, low wetting property caused by hydrophobicity and gelating property in aqueous solution. Therefore, when an unformulated ordinary oral preparation of it such as a capsule filled with an active ingredient is administered,the surface of the preparation is slowly penetrated by water, transformed into gel and finally covered with a viscous plug in the early stage of release. This viscous plug prevents further and rapid penetration of water into the preparation, which results in forming a lump of gel maintaining an original shape of the preparation. Accordingly, the release rate of the active ingredient from a gel layer of the preparation is very slow, which results in low bioavailability. In addition, because the compound of the Chemical Formula 1 has low apparent density and strong electrostatic attraction, agglomeration of particles of the compound occurs. Not only miscibility of the compound of Chemical Formula 1 with various excipients but also fluidity of the mixture is poor during the preparation process. Therefore, there are difficulties to achieve homogenization and reproducibility of the preparation.

Although many trials were made to increase bioavailability of the compound of Chemical Formula 1 by using various pharmaceutical methods, satisfactory results were not obtained due to the physical characteristics that the compound forms gel of hard form by itself in the early stage of release. For example, since solid dispersion method is widely used to increase the dissolution rate of an insoluble active ingredient, there was a trial that the compound of Chemical Formula 1 was dispersed into a water-soluble polymer such as polyvinylpyrrolidone or hydroxypropylmethyl cellulose. However the trial resulted in that the release of the compound of Chemical Formula 1 from the solid dispersion is rather inhibited than improved. It may be the reason for these results that usual the water-soluble polymer used as pharmaceutically inactive carrier such as polyvinylpyrrolidone or hydroxypropylmethyl cellulose rather accelerated than prevented the gelation of the compound of Chemical Formula 1 in the early stage of release.

In addition, there were other trials that the compound of Chemical Formula 1 was formulated into the oral solid preparation, exemplified by tablet or capsule, with crospovidone as a superdisintegrant and/or with various excipients as a common disintegrant, for example, starch, low substituted hydroxypropyl cellulose, microcrystalline cellulose, etc. However the aforementioned trials also failed to achieve satisfactory improvement on dissolution of the compound of Chemical Formula 1.

As the result of the further investigation of the pharmaceutical preparation of the compound of the Chemical Formula 1, including the aforementioned trials, the present inventors have found that the aforementioned carbonate and/or specific disintegrant regionally forms a neutral pH or weakly alkaline environment in the diffusion layer contacting with water during release of the compound of the Chemical Formula 1 or rapidly disperses the preparation, which effectively prevents the gelation caused by hydration in the early stage of release.

In the oral preparation according to the present invention, the compound of Chemical Formula 1 may be used as its pharmaceutically acceptable salt form. Hydrochloric acid, bromic acid, sulfuric acid or phosphoric acid may be used to prepare an inorganic acid salt of the compound of Chemical Formula 1. Citric acid, acetic acid, lactic acid, tartaric acid, fumaric acid, formic acid, propionic acid, oxalic acid, trifluoro acetic acid, methanesulfonic acid, maleic acid, benzoic acid, gluconic acid, glycolic acid, succinic acid, 4-morpholineethanesulfonic acid, camphorsulfonic acid, 4-nitrobenzenesulfonic acid, hydroxy-O-sulfonic acid, 4-toluene sulfonic acid, galacturonic acid, embonic acid, glutamic acid or aspartic acid may be used to prepare an organic acid salt of the compound of Chemical Formula 1. Hydrochloric acid and methanesulfonic acid may be preferably used to prepare an inorganic acid and organic acid salt of the compound of Chemical Formula 1, respectively. In particular, when the salt is prepared by using methanesulfonic acid, 2 methanesulfonic acid salt of the compound of Chemical Formula 1 is preferred.

Although 2 methanesulfonic acid salt of the compound of Chemical Formula 1 has improved water solubility, it still shows a characteristic of an insoluble active ingredient depending on the pH environment of a aqueous solution and gelating property. It is the reason that N-hydroxy-4-{5-[4-(5-isopropyl-2-methyl-1,3-thiazol-4-yl)phenoxy]pentoxy}-benzamidine are dissociated to form the 2 methanesulfonic acid salt of the compound of Chemical Formula 1 when the salt is dissolved in aqueous liquids such as, for example, water, saliva and the gastrointestinal tract after an oral administration. Accordingly, when the aforementioned pharmaceutically acceptable salts of the compound of Chemical Formula 1 are formulated into the oral preparation described in the present invention, the active ingredient is readily released before gelation proceeds in an early stage of release, thereby significantly improving bioavailability of it. Depending on a dose required to show therapeutic effect of the compound, the amount of the compound of Chemical Formula 1 is not particularly limited, but the range of 1˜60% by weight is preferred. A Carbonate used in an oral preparation containing the compound of Chemical Formula 1 is selected from the group consisting of alkali metal carbonate, such as sodium carbonate, potassium carbonate, or the like; alkali metal bicarbonate, such as sodium bicarbonate, potassium bicarbonate, or the like; and alkaline earth metal carbonate such as calcium carbonate, magnesium carbonate, or the like. Sodium bicarbonate or calcium carbonate is preferred.

There were attempts to increase the dissolution rate of an active ingredient using an effervescent reaction alone caused by a simple acid-base neutralization reaction, without any consideration of physicochemical properties of an active ingredient. For example, it was disclosed in Japanese Patent Laid-Open Publication No. 90-704 (corresponding to U.S. Pat. No. 5,091,191) that a compound, of which dissolution profile depends on the pH of aqueous solution, showed pH independent dilution profile, when it was formulated into granules containing mannitol, sodium bicarbonate and a large amount of water-soluble polymer. However, there was described that such an improving effect on the pH dependent dissolution is not caused by using sodium bicarbonate only, but resulted from the combinational interaction of various additives used together.

Unlike those conventional attempts, the present invention is differentiated from them in the aspect that dissolution rate is remarkably improved by not only a simple effervescent reaction but also a various and complicated inhibitory effect on gelation, even though the compound of Chemical Formula 1 and carbonate are formulated into the oral preparation without any other additives such as water-soluble polymer. So, it is apparent that the present invention is distinguished from conventional arts.

That is, the compound of Chemical Formula 1 according to the present invention shows properties of pH dependent solubility and pH dependent gelation. As the pH environment changes from strongly acidic to weakly acidic or weakly alkaline(pH 3˜pH 7.5), properties of solubility and gelation of the compound of Chemical Formula 1 significantly decrease.

Besides, in an oral preparation of the compound of Chemical Formula 1 according to the present invention, carbonate, which inhibits gelation, reacts with gastric juice to produce carbon dioxide in the early stage of release. Consequently, this produced gas causes an oral preparation to be effervescently disintegrated, which results in inhibiting gelation. In addition, the carbonate regionally changes a pH environment of a diffusing layer contacting with water to a neutral or weakly basic condition during the early stage of release, which effectively inhibits the gelation of the compound of Chemical Formula 1.

The carbonate according to the present invention is contained in an amount of about 0.4 to 6.0 parts by weight, preferably 0.5 to 2.0 parts by weight, based on one part by weight of the compound of Chemical Formula 1 or pharmaceutically acceptable salt thereof. When the carbonate is used in an amount of less than 0.4 parts by weight, the release rate of the compound is not enhanced. The carbonate of greater than 6.0 parts by weight generates gas in the gastrointestinal tract and thus may cause abdominal inflation.

An oral preparation of the compound of Chemical Formula 1 according to the present invention comprises one or more disintegrants selected from the group consisting of sodium starch glycolate, carmellose calcium and croscarmellose sodium. Among them, sodium starch glycolate or croscarmellose sodium is preferable. The aforementioned disintegrants rapidly absorb water and extensively swell to disperse active ingredient particles of the compound of Chemical Formula 1 in the early stage of release. So the gelation on the surface of the preparation is effectively inhibited and thus the release from the preparation has increased.

The specific disintegrant that inhibits gelation is contained in an amount of about 0.5˜5.0 parts by weight, based on one part by weight of the compound of Chemical Formula 1 or pharmaceutically acceptable salt thereof. When the disintegrant is contained in an amount of less than 0.5 parts by weight, the improvement effect on the dissolution rate may be decreased, because active ingredients are not evenly dispersed and the inhibitory effect on gelation by carriers is low in the early stage of release. The disintegrant of greater than 5.0 parts by weight does not exhibit an enhancing effect on the release rates of the compound any more, and enlarges the volume of the preparation, thereby causing inconvenience upon ingestion of the oral preparation, which decreases patient compliance.

In order to improve the dissolution rate of the compound of Chemical Formula 1, the compound may be formulated with both the specific disintegrant and carbonate. In the case of combinational use, the dissolution rate is more improved compared with the case of the respective use of the disintegrant or carbonate. Also even when the less amount of the disintegrant and carbonate is used together, the same or more excellent dissolution profile can be obtained. Therefore, it is possible to reduce the volume of an oral preparation because the total amount of the oral preparation can be decreased and large amount of an active ingredient per one dosage unit can be contained therein. Thus, the satisfactory patient compliance could be achieved.

When the disintegrant and carbonate are used together, an oral preparation according to the present invention preferably contains the disintegrant in an amount of about 0.5 to 5.0 parts by weight and the carbonate in an amount of about 0.1 to 6.0 parts by weight, based on one part by weight of the compound of Chemical Formula 1 or pharmaceutically acceptable salt thereof. When the disintegrant and carbonate are used in amounts of less than 0.5 and 0.1 parts by weight, respectively, they do not exhibit a suitable inhibitory effect on gel formation. When the amounts of the disintegrant and carbonate exceed 5.0 and 6. 0 parts by weight, respectively, satisfactory patient compliance is not achieved.

In addition, an oral preparation of the compound of Chemical Formula 1 may further include an excipient. In order to increase the release rate of the active ingredient by effectively inhibiting gel formation and rapidly dispersing the active ingredient, the excipient is preferably an inorganic excipient, such as dibasic calcium phosphate, calcium phosphate, heavy magnesium oxide, precipitated calcium carbonate, or magnesium carbonate. More preferred is dibasic calcium phosphate, calcium phosphate, or heavy magnesium oxide. In contrast, organic excipients, such as microcrystalline cellulose, mannitol, corn starch and lactose, have no enhancing effect on the release rate of the active ingredient.

When the inorganic excipient, such as calcium biphosphate, calcium phosphate or precipitated calcium carbonate, is used in an oral preparation of the compound of Chemical Formula 1 according to the present invention, it enhances the dissolution rate and bioavailability, and acts as a calcium supplier. Accordingly, from this point of view, the oral preparation, which contains the compound of Chemical Formula 1 and the aforementioned inorganic excipients can be expected to exhibit synergy effect on prevention and treatment of osteoporosis.

In addition to the aforementioned components, the present preparation may include a pharmaceutically acceptable ordinary excipient or adjuvant, and may be formulated into a solid formulation for oral administration, such as tablets, capsules, granules, or fine granules, through an ordinary pharmaceutical method.

That is, according to the present invention, the present composition may be formulated as granules, and may be supplemented with a lubricant and other pharmaceutically acceptable additives and directly filled into hard capsules in a powder or granule form. Otherwise, the composition may be supplemented with pharmaceutical additives for tabletting and compressed to produce tablets according to a known method.

The oral preparation according to the present invention may further include a pharmaceutically acceptable ordinary additive. Examples of the additive include binders, lubricants, glidants, surfactants, colorants and taste/smell masking agents. Pharmaceutically acceptable ordinary binders and glidants are available. The binders are exemplified by maltose, arabia gum and hydroxypropylcellulose. The lubricants are exemplified by carnauba wax, light anhydrous silic acid, synthetic aluminum silicate, stearic acid, magnesium stearate and talc.

Widely known wet granulation methods may be used for the granulation of the oral preparation according to the present invention. The compound of Chemical Formula 1 or pharmaceutically acceptable salt thereof is mixed with a carbonate and/or specific disintegrant that inhibits gelation, and, if necessary, with a pharmaceutically acceptable ordinary excipients or additives. The mixture thus obtained is wet granulated with solution, which had been prepared by dissolving a binder in a solvent such as ethanol or isopropanol, etc., or in a mixed solvent thereof. Then, granulation is carried out through a stirring granulator or a high speed stirring granulator.

As another granulation method for the oral preparation according to the present invention, the aforementioned mixture is wet massed with a binder solution, kneaded, granulated by an extrusion granulator and screened.

As another granulation method for the oral preparation according to the present invention, the aforementioned mixture is granulated with spraying a binder solution under a fluidized bed granulator.

The dosage form of the oral preparation according to the present invention may depend on patient's weight, age, gender, health state, diet, administration period, administration route, excretion rate, severity of a illness, and the like. 2 methanesulfonic acid salt of the compound of Chemical Formula 1 may be administrated, for example, in a daily dosage of 1 to 1,000 mg/kg, preferably 10 to 500 mg/kg. The daily dosage may be divided into one to several doses.

### Mode for Invention

Hereinafter, preferred example embodiments of the present invention will be described more fully to facilitate understanding of the invention. This invention may, however, be embodied in many different forms and should not be construed as limited to the example embodiments set forth herein.

### Reference Example 1

### Preparation of 2 Methanesulfonic Acid Salt of the Compound

2 methanesulfonic acid salt of the compound of Chemical Formula 1 according to the present invention was prepared by the following method.

150 g (0.33 mol) of N-hydroxy-4-{5-[4-(5-isopropyl-2-methyl-1,3-thiazol-4-yl)phenoxy]pentoxy}-benzamidine was dissolved in 1.1 L of ethanol, mixed with 47 mL (2.2 equivalents) of methanesulfonic acid with dropping, and subsequently stirred at room temperature for 1 hr. The solution thus obtained was then mixed with 3 L of acetone and 1.1 L of n-hexane, and subsequently stirred for further 1 hour. The solid thus produced was recovered by filtration, washed with acetone, and dried under vacuum. As a result, 188 g (yield: 88%) of N-hydroxy-4-{5-[4-(5-isopropyl-2-methyl-1,3-thiazol-4-yl)phenoxy]pentoxy}-benzamidine 2 methanesulfonic acid salt was obtained as a white solid.

Melting point: 156.4° C.

### Reference Example 2

### Gelation Experiment

The following test was carried out to evaluate the degree of gelation depending on the concentration of 2 methanesulfonic acid salt of the compound of Chemical Formula 1 according to the present invention.

200 mg of 2 methanesulfonic acid salt of the compound of Chemical Formula 1 was dissolved in 10 mL of water (20 mg/mL). The solution was diluted with water to give 20, 10, 5, and 2.5 mg/mL. Viscosity of these diluted solutions was measured according to the following test conditions described in Table 1, and also the results for the test are shown in Table 1.

TABLE 1

| | Concentration | Viscosity |
|---|---|---|
| Test conditions | (mg/ml) | (cP) |
| (a) Instrument: Brookfield digital | 2.5 | 6.34 |
| viscometer DV-II+ | 5 | 11.9 |
| (b) Temperature: 10° C. ± 0.3° C. | 10 | 20.0 |
| (c) Spindle: S 51 | 20 | 78.9 |

As shown in Table 1, it has been observed that 2 methanesulfonic acid salt of the compound of Chemical Formula 1 exhibited high gelation property in aqueous solution. The results say that viscosity of the solution significantly increased when the concentration of the compound increased. The compound of Chemical Formula 1 and other salt thereof showed the similar gelation property.

### Examples 1 to 24

### Preparation of Capsule

2 methanesulfonic acid salt, hydrochloric acid salt or free base of the compound of Chemical Formula 1 was mixed with a carbonate or a specific disintegrant or both of the two. Also, if necessary, other excipients were added to the mixture. The mixture was moistened with a binder solution, which had been prepared by dissolving polyvinylpyrrolidone in ethanol, isopropanol, or the like, or mixture thereof. The wet mass was, kneaded, passed through a 16 mesh screen, dried at 50° C. and screened through a 25 mesh sieve. The granules thus obtained were filled into a gelatin capsule in an amount of 200 mg as an active ingredient using a capsule filler.

The content ratio of the compositions of Examples 1 to 6, in which 2 methanesulfonic acid salt of the compound of Chemical Formula 1 and a carbonate are contained, is shown in Table 2. The content ratio of the compositions of Examples 13 to 18, in which 2 methanesulfonic acid salt of the compound of Chemical Formula 1, a carbonate and disintegrant are contained, is shown in Table 4.

The content ratio of the compositions of Examples 19 to 24, in which Hydrochloric acid salt or free base of the compound of Chemical Formula 1 was mixed with a carbonate or a disintegrant, or both of the two, is shown in Table 5.

TABLE 2

| | Example (granule, mg) | | | | | |
|---|---|---|---|---|---|---|
| | 1 | 2 | 3 | 4 | 5 | 6 |
| 2 methanesulfonic acid salt of | 100 | 100 | 100 | 100 | 100 | 100 |
| the compound of Chemical | | | | | | |
| Formula 1 | | | | | | |
| Sodium bicarbonate | 100 | | | | | 40 |
| Calcium carbonate | | 100 | | | | |
| Potassium carbonate | | | 100 | | | |
| Sodium carbonate | | | | 100 | | |
| Potassium bicarbonate | | | | | 100 | |
| Lactose | 50 | 50 | 50 | 50 | 50 | |
| Calcium biphosphate | | | | | | 110 |
| Polyvinylpyrrolidone | 4 | 4 | 4 | 4 | 4 | 4 |

TABLE 3

| | Example (granule, mg) | | | | | |
|---|---|---|---|---|---|---|
| | 7 | 8 | 9 | 10 | 11 | 12 |
| 2 methanesulfonic acid salt of | 100 | 100 | 100 | 100 | 100 | 100 |
| the compound of Chemical | | | | | | |
| Formula 1 | | | | | | |
| Sodium starch glycolate | 100 | | | 50 | 80 | |
| Croscarmellose sodium | | 100 | | | | 50 |
| Carmellose calcium | | | 100 | | | |
| Lactose | | | | | | 50 |
| Mannitol | | | | | 20 | |
| Calcium biphosphate | | | | 50 | | |
| Polyvinylpyrrolidone | 4 | 4 | 4 | 4 | 4 | 4 |

TABLE 4

| | Example (granule, mg) | | | | | |
|---|---|---|---|---|---|---|
| | 13 | 14 | 15 | 16 | 17 | 18 |
| 2 methanesulfonic acid salt of | 100 | 100 | 100 | 100 | 100 | 100 |
| the compound of Chemical | | | | | | |
| Formula 1 | | | | | | |
| Sodium starch glycolate | 50 | | | 100 | | |
| Croscarmellose sodium | | 50 | | | 100 | |
| Carmellose calcium | | | 50 | | | 100 |
| Sodium bicarbonate | 10 | | | 20 | | |
| Calcium carbonate | | 10 | | | 20 | |
| Potassium bicarbonate | | | 10 | | | 20 |
| Polyvinylpyrrolidone | 4 | 4 | 4 | 4 | 4 | 4 |

TABLE 5

| | Example (granule, mg) | | | | | |
|---|---|---|---|---|---|---|
| | 19 | 20 | 21 | 22 | 23 | 24 |
| Hydrochloric acid salt of the | 100 | 100 | 100 | | | |
| compound of Chemical | | | | | | |
| Formula 1 | | | | | | |
| Free base of the compound of | 50 | | | 100 | 100 | 100 |
| Chemical Formula 1 | | | | | | |
| Sodium starch glycolate | 100 | | 50 | 100 | | 50 |
| Sodium bicarbonate | | 100 | 10 | | 100 | 10 |
| Lactose | | 50 | | | 50 | |
| Polyvinylpyrrolidone | 4 | 4 | 4 | 4 | 4 | 4 |

### Example 25 to 30

### Preparation of Tablet

2 methanesulfonic acid salt, hydrochloric acid salt or free base of the compound of Chemical Formula 1 was mixed with a carbonate or a specific disintegrant or both of the two. Also, if necessary, other excipients were added to the mixture. The mixture was moistened with a binder solution, which had been prepared by dissolving polyvinylpyrrolidone in ethanol, isopropanol or the like, or mixture thereof. The wet mass was kneaded, passed through a 16 mesh screen, dried at 50° C. and screened through a 25 mesh sieve. The granules thus obtained were mixed with magnesium stearate, and compressed to tablet, which contained 100 mg as 2 methanesulfonic acid salt of the compound of Chemical Formula 1, by using a conventional tabletting machine. The hardness of the tablet was in the range of 4˜5 KP.

The content ratio of the compositions of Examples 25 to 30, in which 2 methanesulfonic acid salt, hydrochloric acid salt or free base of the compound of Chemical Formula 1 was mixed with a carbonate, a specific disintegrant or both of the two, is shown in Table 6. The content ratio of the compositions of Examples 31 to 36, in which 2 methanesulfonic acid salt of the compound of Chemical Formula 1 was mixed with a specific disintegrant; and on organic excipient, such as microcrystalline cellulose, cornstarch or lactose; or on inorganic excipient, such as heavy magnesium oxide, calcium biphosphate, or calcium phosphate, is shown in Table 7.

TABLE 6

| | Example (mg) | | | | | |
|---|---|---|---|---|---|---|
| | 25 | 26 | 27 | 28 | 29 | 30 |
| 2 methanesulfonic acid salt of | 100 | 100 | 100 | | | |
| the compound of Chemical | | | | | | |
| Formula 1 | | | | | | |
| Hydrochloric acid salt of the | | | | 100 | 100 | |
| compound of Chemical | | | | | | |
| Formula 1 | | | | | | |
| Free base of the compound of | | | | | | 100 |
| Chemical Formula 1 | | | | | | |
| Sodium starch glycolate | 100 | | 70 | 100 | | 100 |
| Sodium bicarbonate | | 100 | 15 | | 100 | |
| Lactose | | 50 | | | 50 | |
| Polyvinylpyrrolidone | 4 | 4 | 4 | 4 | 4 | 4 |
| Magnesium stearate | 7 | 8 | 6 | 7 | 8 | 7 |

TABLE 7

| | Example(mg) | | | | | |
|---|---|---|---|---|---|---|
| | 31 | 32 | 33 | 34 | 35 | 36 |
| 2 methanesulfonic acid salt of | 100 | 100 | 100 | 100 | 100 | 100 |
| the compound of Chemical | | | | | | |
| Formula 1 | | | | | | |
| Sodium starch glycolate | 80 | 80 | 80 | 80 | 80 | 80 |
| Micro crystalline cellulose | 160 | | | | | |
| Cornstarch | | 160 | | | | |
| Lactose | | | 160 | | | |
| Heavy magnesium oxide | | | | 160 | | |
| Calcium biphosphate | | | | | 160 | |
| Calcium phosphate | | | | | | 160 |
| Povidone | 12 | 12 | 12 | 12 | 12 | 12 |
| Magnesium stearate | 12 | 12 | 12 | 12 | 12 | 12 |

### Comparative Examples 1-18

### Preparation of Capsule or Tablet

Raw materials of 2 methanesulfonic acid salt, hydrochloric acid salt or free base of the compound of Chemical Formula 1 according to the present invention was individually sieved through a 45 mesh sieve and filled into a gelatin capsule in an amount of 200 mg of active ingredient (Comparative Examples 1,10 and 13).

2 methanesulfonic acid salt, hydrochloric acid salt or free base of the compound of Chemical Formula 1 was mixed with a small amount of a carbonate or other excipients. After Granules were prepared according to the same method applied to examples, capsules (Comparative Examples 2 to 9, 11, 12, 14 and 15) and tablets (Comparative Examples 16 to 18) were prepared by using a capsule filler and a conventional tabletting machine, respectively. The content ratio of the compositions is shown in Tables 8, 9 and 10.

TABLE 8

| | Comparative | | | | | |
|---|---|---|---|---|---|---|
| | Example (mg) | | | | | |
| | 1 | 2 | 3 | 4 | 5 | 6 |
| 2 methanesulfonic acid salt of | 100 | 100 | 100 | 100 | 100 | 100 |
| the Compound of Chemical | | | | | | |
| formula 1 | | | | | | |
| Crospovidone | | 100 | | | | |
| Low substituted cellulose | | | 100 | | | |
| Micro crystalline cellulose | | | | 100 | | |
| Cornstarch | | | | | 100 | |
| Lactose | | | | | | 100 |
| Polyvinylpyrrolidone | | 4 | 4 | 4 | 4 | 4 |

TABLE 9

| | Comparative | | | | | |
|---|---|---|---|---|---|---|
| | Example (mg) | | | | | |
| | 7 | 8 | 9 | 10 | 11 | 12 |
| 2 methanesulfonic acid salt of | 100 | 100 | 100 | | | |
| the compound of Chemical | | | | | | |
| Formula 1 | | | | | | |
| Hydrochloric acid salt of the | | | | 100 | 100 | 100 |
| compound of Chemical | | | | | | |
| Formula 1 | | | | | | |
| Sodium bicarbonate | 10 | | | | | 10 |
| Calcium carbonate | | 10 | | | | |
| Potassium carbonate | | | 10 | | | |
| Crospovidone | | 50 | | | 100 | |
| Lactose | 140 | 140 | 140 | | 50 | 140 |
| Polyvinylpyrrolidone | 4 | 4 | 4 | | 4 | 4 |

TABLE 10

| | Comparative | | | | | |
|---|---|---|---|---|---|---|
| | Example (mg) | | | | | |
| | 13 | 14 | 15 | 16 | 17 | 18 |
| 2 methanesulfonic acid salt of | | | | 100 | 100 | 100 |
| the compound of Chemical | | | | | | |
| Formula 1 | | | | | | |
| Free base of the compound of | 100 | 100 | 100 | | | |
| Chemical Formula 1 | | | | | | |
| Sodium bicarbonate | | | 10 | | | 10 |
| Crospovidone | | 100 | | 100 | | |
| Low substituted cellulose | | | | | 100 | |
| Lactose | | 50 | 140 | 50 | 50 | 140 |
| Polyvinylpyrrolidone | | 4 | 4 | 4 | 4 | 4 |
| Magnesium stearate | | | | 8 | 8 | 8 |

### Experimental Example 1

### Dissolution Studies

In-vitro dissolution studies were performed on capsules and tablets prepared in Examples 1 to 36 and Comparative Examples 1 to 18. The medium was 900 ml of 0.1 N HCl at 37° C. in Apparatus 2 (USP 27,<711> Dissolution, pp2303˜2304) (paddle, 50 rpm)

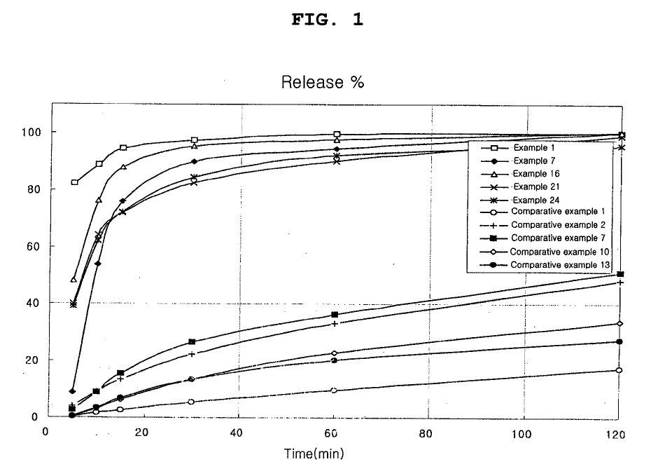

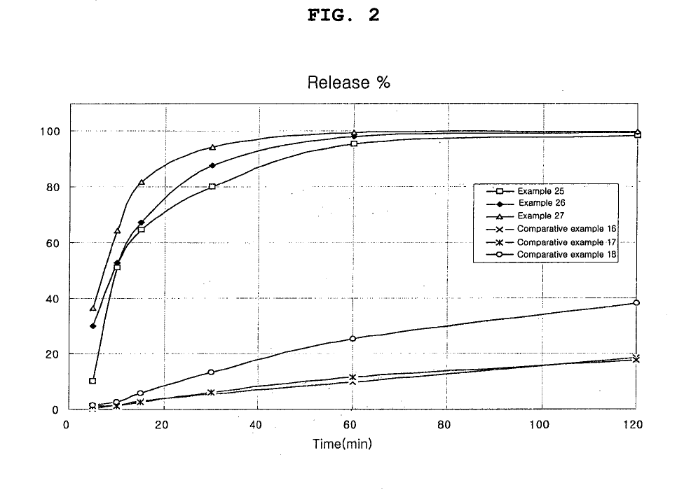

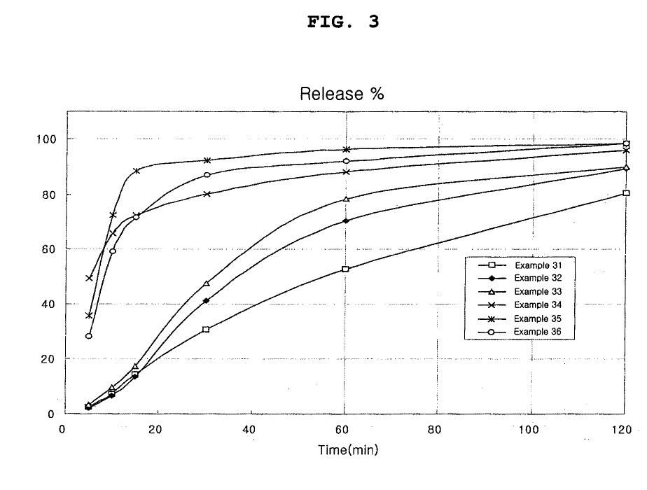

3 mL of the test medium was drawn out at the predetermined intervals (5, 10, 15, 30, 60 and 120 minutes) and 3 mL of a fresh meium preheated to 37° C. was newly supplemented. Immediately after being drawn out, the sample were centrifuged and filtered through a membrane filter with pore size of 0.45 μm. The amount 5 (% released) of the active ingredient dissolved in the test medium was determined by measuring its absorbance at 254 nm with UV spectrometer. The results are shown in Tables 11, 12 and FIGS. 1 to 3.

TABLE 11

| | Release % | | | | | |
|---|---|---|---|---|---|---|
| | 5 min. | 10 min. | 15 min. | 30 min. | 60 min. | 120 min. |
| Example 1 | 82.3 | 88.8 | 94.4 | 97.5 | 99.7 | 99.9 |
| Example 2 | 61.3 | 76.8 | 81.4 | 87.7 | 91.7 | 92.1 |
| Example 3 | 0.6 | 4.4 | 9.7 | 35.8 | 79.2 | 89.2 |
| Example 4 | 1.4 | 11.9 | 31.4 | 73.8 | 92.1 | 94.8 |
| Example 5 | 9.8 | 18.1 | 27.5 | 54.5 | 83.1 | 89.7 |
| Example 6 | 33.3 | 48.2 | 58.8 | 72.2 | 84.5 | 89.5 |
| Example 7 | 9.0 | 53.9 | 75.8 | 89.9 | 94.5 | 100.3 |
| Example 8 | 7.2 | 42.3 | 70.2 | 85.4 | 92.8 | 99.8 |
| Example 9 | 5.1 | 38.6 | 62.3 | 78.3 | 85.2 | 94.5 |
| Example 10 | 2.5 | 11.6 | 31.5 | 90.5 | 99.1 | 99.8 |
| Example 11 | 5.2 | 12.8 | 23.7 | 40.0 | 66.0 | 85.3 |
| Example 12 | 3.3 | 13.3 | 26.4 | 51.9 | 64.7 | 74.0 |
| Example 13 | 43.2 | 69.3 | 80.2 | 89.1 | 92.3 | 96.6 |
| Example 14 | 24.6 | 57.3 | 70.5 | 80.9 | 84.1 | 89.4 |
| Example 15 | 27.5 | 61.0 | 68.8 | 76.8 | 82.3 | 88.0 |
| Example 16 | 48.0 | 76.3 | 87.8 | 95.5 | 97.7 | 100.2 |
| Example 17 | 60.9 | 77.5 | 86.0 | 95.7 | 97.4 | 98.1 |
| Example 18 | 53.3 | 70.7 | 82.0 | 90.1 | 94.3 | 99.6 |
| Example 19 | 8.9 | 35.8 | 53.2 | 81.2 | 91.5 | 101.4 |
| Example 20 | 35.0 | 58.2 | 68.2 | 78.7 | 88.1 | 95.2 |
| Example 21 | 40.2 | 64.3 | 71.8 | 82.6 | 90.1 | 99.2 |
| Example 22 | 5.5 | 25.1 | 42.1 | 72.8 | 88.9 | 95.5 |
| Example 23 | 30.1 | 45.5 | 57.2 | 76.1 | 90.9 | 100.5 |
| Example 24 | 39.2 | 62.1 | 72.3 | 84.4 | 92.1 | 95.8 |
| Example 25 | 10.2 | 50.9 | 64.5 | 80.0 | 95.3 | 98.4 |
| Example 26 | 29.9 | 52.6 | 67.2 | 87.7 | 98.0 | 99.4 |
| Example 27 | 36.3 | 64.4 | 81.8 | 94.4 | 99.5 | 99.9 |
| Example 28 | 11.6 | 51.0 | 63.9 | 80.3 | 94.1 | 96.9 |
| Example 29 | 31.6 | 56.5 | 68.4 | 89.6 | 100.2 | 100.5 |
| Example 30 | 10.5 | 49.0 | 60.8 | 78.4 | 93.8 | 95.8 |
| Example 31 | 2.5 | 7.2 | 14.4 | 30.7 | 52.6 | 80.4 |
| Example 32 | 2.1 | 6.5 | 13.4 | 41.3 | 70.2 | 89.6 |
| Example 33 | 3.2 | 9.7 | 17.3 | 47.6 | 78.4 | 90.2 |
| Example 34 | 49.3 | 65.7 | 72.4 | 80.1 | 88.4 | 96.2 |
| Example 35 | 35.8 | 72.4 | 88.6 | 92.7 | 96.6 | 98.6 |
| Example 36 | 28.2 | 59.3 | 71.4 | 87.2 | 92.3 | 98.4 |

TABLE 12

| | Release % | | | | | |
|---|---|---|---|---|---|---|
| | 5 min. | 10 min. | 15 min. | 30 min. | 60 min. | 120 min. |
| Comparative | 0.3 | 1.6 | 2.5 | 5.3 | 9.8 | 17.6 |
| Example 1 | | | | | | |
| Comparative | 4.0 | 9.3 | 13.6 | 22.3 | 33.2 | 48.1 |
| Example 2 | | | | | | |
| Comparative | 1.0 | 1.5 | 2.3 | 4.2 | 8.2 | 13.5 |
| Example 3 | | | | | | |
| Comparative | 0.8 | 3.0 | 8.0 | 17.9 | 29.5 | 38.2 |
| Example 4 | | | | | | |
| Comparative | 1.1 | 3.6 | 8.5 | 16.2 | 31.2 | 42.8 |
| Example 5 | | | | | | |
| Comparative | 0.3 | 1.6 | 3.1 | 7.4 | 13.4 | 22.2 |
| Example 6 | | | | | | |
| Comparative | 2.7 | 8.9 | 15.4 | 26.7 | 36.5 | 51.1 |
| Example 7 | | | | | | |
| Comparative | 1.6 | 5.4 | 11.0 | 23.4 | 30.8 | 41.0 |
| Example 8 | | | | | | |
| Comparative | 1.2 | 2.8 | 7.6 | 12.9 | 23.1 | 39.8 |
| Example 9 | | | | | | |
| Comparative | 0.6 | 3.1 | 6.2 | 13.5 | 22.9 | 33.7 |
| Example 10 | | | | | | |
| Comparative | 0.6 | 1.4 | 2.7 | 6.2 | 10.5 | 17.9 |
| Example 11 | | | | | | |
| Comparative | 2.1 | 7.8 | 12.3 | 20.1 | 29.8 | 40.7 |
| Example 12 | | | | | | |
| Comparative | 0.5 | 3.5 | 7.0 | 13.5 | 20.3 | 27.5 |
| Example 13 | | | | | | |
| Comparative | 0.7 | 2.1 | 3.3 | 7.8 | 15.5 | 22.9 |
| Example 14 | | | | | | |
| Comparative | 2.2 | 5.5 | 10.4 | 18.8 | 27.5 | 35.1 |
| Example 15 | | | | | | |
| Comparative | 0.3 | 1.5 | 2.8 | 5.5 | 9.8 | 19.7 |
| Example 16 | | | | | | |
| Comparative | 1.1 | 1.3 | 2.5 | 6.1 | 11.5 | 17.8 |
| Example 17 | | | | | | |
| Comparative | 1.6 | 2.5 | 5.8 | 13.5 | 25.2 | 38.2 |
| Example 18 | | | | | | |

As shown in Tables 11 and 12, it has been observed that the capsules and tablets of the present invention showed a remarkably higher dissolution rate compared with that of capsules and tablets prepared in Comparative Examples. It cane explained that the specific disintegrant according to the present invention rapidly absorbed water, enormously swelled, effectively dispersed an active ingredient and subsequently inhibited the gelation in an early stage of release, thereby resulting in enhancing the release rate.

In contrast, in the cases of Comparative Examples 1, 10, and 13 (without gelation inhibitor), capsules filled with an active ingredient slowly absorbed water to form a gel which, eventually, agglomerated with gelatin capsule. Even when 20 minutes elapsed, the released amount of active ingredient was very low and the gelatin capsule was not removed completely. The active ingredient was slowly released from the gel layer with maintaining a dosage form.

There was no significant improvement in the dissolution rate when formulated with crospovidone, which is commonly used as a superdisintegrant in the formulation of an oral preparation, or other excipients, which are commonly used as a conventional disintegrator and exemplified as low-substituted hydroxypropyl cellulose, micro crystalline cellulose, cornstarch, or lactose (Comparative Examples 2 to 6).

When the carbonate was contained (Examples 1 to 6), tablets were abruptly disintegrated through an effervescent reaction, and rapidly released an active ingredient. This maybe understood that the carbohydrate effectively prevented gelation in an early stage of release, which resulted in enhancing the release rate, because abrupt effervescent reaction by a carbonate rapidly dispersed granule particles, burst a gelatin capsule and regionally changed the pH environment of diffusion layer, from which an active ingredient is released, to weakly acidic or weakly basic condition.

When the oral preparation additionally included a pharmaceutically acceptable excipients, it has been observed that an inorganic excipient (Examples 34 to 36) such as calcium biphosphate was more effective on the dissolution of 2 methanesulfonic acid salt of the compound of Chemical Formula 1 than an organic excipient (Examples 31 to 33) such as lactose. It may be explained that the inorganic excipient evenly existing between the molecules of 2 methanesulfonic acid salt of the compound of Chemical Formula 1 contributed to rapid dispersion of an active ingredient by effective inhibiting gelation during the dissolution. In other words, it may be said that, in an oral solid dosage form of the compound of Chemical Formula 1 according to the present invention, the inorganic excipient assists the action of a carbonate and a specific disintegrant in an early stage of release.

When hydrochloric acid salt of the compound of Chemical Formula 1 was granulated with a carbonate, a specific disintegrant, or both of the two (Examples 19 to 21), the dissolution rate was remarkably increased, compared with hydrochloric acid salt of the compound of Chemical Formula 1 (Comparative Example 10).

When free base of the compound of Chemical Formula 1 was granulated with a carbonate, a specific disintegrant or both of the two (Examples 22 to 24), the dissolution rate was remarkably increased compared with free base of the compound of Chemical Formula 1 (Comparative Example 13).

In addition, when a carbonate and a specific disintegrant were simultaneously used together, but even in a small amount, equal or more excellent dissolution profiles were achieved compared with the case of single use.

In conclusion, rapid dissolution can be achieved when the compound of Chemical Formula 1 was formulated with a carbonate and/or disintegrant which effectively inhibits gelation before the agglomeration of the compound in the early stage of release.

### Experimental Example 2

### Pharmacokinetic Studies

The following experiments were carried out to evaluate the bioavailability of capsules prepared in Example 16 and Comparative Example 1.

1) Experiment Animal

Male beagle dogs were supplied from Jung Ang Lab Animal Inc. The weight of animals used for pharmacokinetic studies was in a range of 7.6˜10.5 kg. The dogs were acclimated in a laboratory for at least 1 week before administration.

2) Administration

Animals were fasted overnight through 8 hours before the experiment. The capsules of Example 16 and Comparative Example 1 were administered orally. The administered amount of the compound of Chemical Formula 1 was 50 mg per 1 kg of an animal.

3) Blood Collection and Analysis

After the oral administration of each capsule, the concentration of an active ingredient in plasma was measured by the following method. For pharmacokinetic studies, blood was collected from cephalic vein of the beagle dog at predetermined intervals (0, 0.5, 1, 1.5, 2, 3, 5, 8 and 24 hours post-dosing) and plasma was separated immediately after blood collection and stored at −20° C. till assay. For HPLC analysis of the compound of Chemical Formula 1, each sample was thawed to room temperature and mixed with equal volume of internal standard solution (containing 30 μg/ml of betamethasone in acetonitrile). The mixture was stirred for 1 minute by a shaking apparatus and centrifuged for 10 minutes at 12,000 rpm. The aliquots of supernatant were injected into the HPLC for quantitation (Waters Module 1).

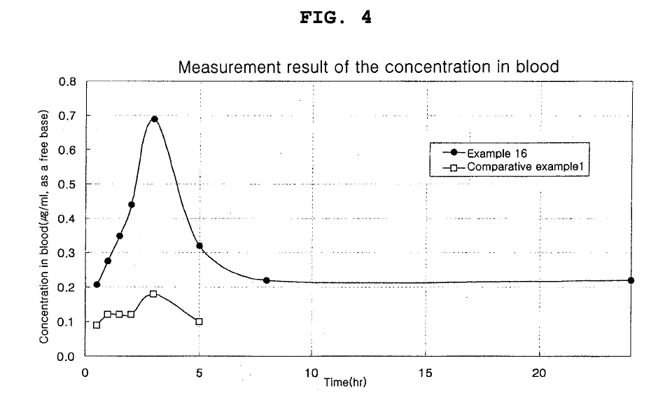

The concentrations of the compound of the Chemical Formula 1 in plasma after oral administration are shown in Table 13 and FIG. 4. All pharmacokinetic parameters are shown in Table 14.

TABLE 13

Concentration of the
compound of Chemical Formula
1 in plasma (μg/ml, n = 3)

| Time (hr) | Example 16 | Comparative Example 1 |
|---|---|---|
| 0.5 | 0.21 ± 0.01 | 0.09 |
| 1.0 | 0.28 ± 0.01 | 0.12 ± 0.01 |
| 1.5 | 0.35 ± 0.04 | 0.12 ± 0.02 |
| 2.0 | 0.44 ± 0.06 | 0.12 ± 0.01 |
| 3.0 | 0.69 ± 0.16 | 0.18 ± 0.11 |
| 5.0 | 0.32 ± 0.05 | 0.10 ± 0.01 |
| 8.0 | 0.22 ± 0.17 | — |
| 24.0 | 0.22 ± 0.01 | — |

TABLE 14

Pharmacokinetic parameters
of the compound of
Chemical Formula 1

| | Example 16 | Comparative Example 1 |
|---|---|---|
| Cₘₐₓ (μg/ml) | 0.69 ± 0.16 | 0.19 ± 0.10 |
| Tₘₐₓ (hr) | 3.00 ± 0.00 | 2.67 ± 0.58 |
| Half life (hr) | 3.61 ± 1.17 | unmeasurable |
| AUC₀₋ₜ (μg · hr/ml) | 2.89 ± 0.33 | 0.61 ± 0.16 |
| Total | 4.03 ± 0.12 | unmeasurable |
| AUC (±g · hr/ml) | | |
| Cₘₐₓ ratio | 3.6 | 1 |
| AUC₀₋ₜ ratio | 4.7 | 1 |

Cₘₐₓ : Maximum concentration in plasma
Tₘₐₓ : Time required to reach the maximum concentration in plasma
AUC₀₋ₜ : area under the curve of concentration in plasma vs time 0 to t
Total AUC : area under the curve of concentration in plasma vs time 0 to ∞

As shown in Table 14 and FIG. 4, in the case of a raw material of 2 methanesulfonic acid salt of the compound of Chemical Formula 1 (Comparative Example 1), there was no apparent change in concentrations in plasma as time passed.

As shown in Table 14, when the capsule (Example 16) prepared according to the present invention was orally administered to beagle dogs, Cₘₐₓ and AUC₀₋ₜ increased to 3.6 folds and 4.7 folds, respectively, compared with that of the raw material of 2 methanesulfonic acid salt of the compound of Chemical Formula 1 (Comparative Example 1).

### INDUSTRIAL APPLICABILITY

An oral preparation according to the present invention improves dissolution rate and bioavailibility of N-hydroxy-4-{5-[4-(5-isopropyl-2-methyl-1,3-thiazol-4-yl)phenoxy]pentoxy}-benzamidine or pharmaceutically acceptable salt thereof by inhibiting gelation of those while contacting with water in the early stage of release. Therefore, the oral preparation according to the present invention may be very usefully utilized for pharmaceutical industries.

1. An oral preparation comprising:
a compound of Chemical Formula 1 or pharmaceutically acceptable salt thereof; and
one or more carbonates selected from the group consisting of an alkali metal carbonate, an alkali metal bicarbonate, and an alkaline earth metal carbonate.

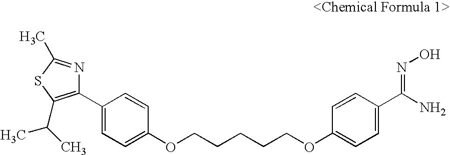

2. An oral preparation comprising:
the compound of Chemical Formula 1 of claim 1 or pharmaceutically acceptable salt thereof; and
one or more disintegrants selected from the group consisting of sodium starch glycolate, carmellose calcium and croscarmellose sodium.

3. An oral preparation comprising:
the compound of Chemical Formula 1 of claim 1 or pharmaceutically acceptable salt thereof;
one or more carbonates selected from the group consisting of alkali metal carbonate, alkali metal bicarbonate, and alkaline earth metal carbonate; and
one or more disintegrants selected from the group consisting of sodium starch glycolate, carmellose calcium and croscarmellose sodium.

4. The oral preparation as claimed in any of claims 1 to 3, wherein the pharmaceutically acceptable salt is 2 methanesulfonic acid salt or hydrochloric acid salt.

5. The oral preparation of claim 1, wherein the carbonate is contained in an amount of about 0.4 to 6.0 parts by weight based on one part by weight of the compound of Chemical Formula 1 or pharmaceutically acceptable salt thereof.

6. The oral preparation of claim 3, wherein the carbonate is contained in an amount of about 0.1 to 6.0 parts by weight based on one part by weight of the compound of Chemical Formula 1 or pharmaceutically acceptable salt thereof.

7. The oral preparation as claimed in any of claims 5 and 6, wherein the carbonate is sodium bicarbonate or calcium carbonate.

8. The oral preparation as claimed in any of claims 2 and 3, wherein the disintegrant is contained in an amount of about 0.5 to 5.0 parts by weight based on one part by weight the compound of Chemical Formula 1 or pharmaceutically acceptable salt thereof.

9. The oral preparation of claim 8, wherein the disintegrant is sodium starch glycolate or croscarmellose sodium.

10. The oral preparation as claimed in any of claims 1 to 3, wherein the oral preparation is a formulation selected from the group consisting of a tablet, capsule, granule, and fine granule.

11. The oral preparation as claimed in any of claims 1 to 3, wherein the oral preparation comprises calcium biphosphate, calcium phosphate, precipitated calcium carbonate or heavy magnesium oxide as an inorganic excipient to improve dissolution rate.

12. The oral preparation of claim 11, wherein the inorganic excipients, such as calcium biphosphate, calcium phosphate or precipitated calcium carbonate, are used for the prevention and treatment of osteoporosis together with the compound of Chemical Formula 1 of claim 1 or pharmaceutically acceptable salt thereof by acting as a calcium supplier in the body.
